(19) United States
(12) Patent Application Publication
(10) Pub. No.: US 2018/0106708 A1
(43) Pub. Date: Apr. 19, 2018

(54) HYDRAULIC FRACTURABILITY INDEX USING HIGH RESOLUTION CORE MEASUREMENTS

(71) Applicant: Schlumberger Technology Corporation, Sugar Land, TX (US)

(72) Inventors: Eduard Siebrits, Salt Lake City, UT (US); Sonia Marino, Salt Lake City, UT (US); Roberto Suarez-Rivera, Salt Lake City, UT (US); Joel Wesley Martin, Farmington, UT (US); Upul Samarasingha, South Jordan, UT (US); Chaitanya Deenadayalu, Salt Lake City, UT (US)

(21) Appl. No.: 15/573,540

(22) PCT Filed: Nov. 5, 2015
(86) PCT No.: PCT/US2015/059279

§ 371 Date: Nov. 13, 2017

Related U.S. Application Data
(60) Provisional application No. 62/164,107, filed on May 20, 2015.

(51) Int. Cl. G01N 3/46; G01N 23/046; E21B 43/267; G06G 7/50

(52) U.S. Cl. CPC G01N 3/46; G01N 23/046; G01N 33/24; G06G 7/50; E21B 43/267

(57) ABSTRACT

A workflow is provided that characterizes the hydraulic fracturability of a rock based on properties obtained from CT scanning and from non-CT based data. The characterization is based on obtaining a plurality of properties of a core sample as a function of axial location in the core sample. The workflow includes obtaining CT data from at least one CT scan of the core, obtaining heterogeneity data of the core, generating a heterogeneous rock analysis (HRA) model based at least on the obtained CT data and heterogeneity data; quantifying statistically significant distinct rock classes in the core, and assigning hydraulic fracturability index (HFI) values to each distinct rock class, as well as any HFI variation within each rock class. An HFI value is assigned to each rock class, and within a rock class, in the core and those values can be propagated to other locations in the same or surrounding wells.

### CROSS-REFERENCE TO RELATED APPLICATION

This application claims priority from U.S. Provisional Application 62/164,107, filed May 20, 2015, which is incorporated herein by reference.

### BACKGROUND

### Field

The present disclosure relates to measurements of rock core samples.

### Related Art

A reservoir is a subsurface body of rock having sufficient porosity and permeability to store and transmit fluids, such as hydrocarbons, including natural gas and petroleum. Sedimentary rocks are the most common reservoir rocks because they have more porosity than most igneous and metamorphic rocks and form under temperature conditions at which hydrocarbons can be preserved. A reservoir is a critical component of a complete petroleum system.

An “unconventional resource” is an umbrella term for oil and natural gas that is produced by means that do not meet the criteria for conventional production. At present, the term is used in reference to oil and gas reservoirs whose porosity, permeability, fluid trapping mechanism, or other characteristics differ from conventional sandstone and carbonate reservoirs. Coalbed methane, gas hydrates, shale gas, fractured reservoirs, and tight gas sands are considered unconventional resources. Unconventional reservoirs (i.e., reservoirs of unconventional resources), while geographically extensive and exhibiting relatively simple structural architecture and stratigraphic continuity, can be composed of a number of lithofacies that change in thickness, regional distribution, and stacking patterns. While this makes unconventional reservoirs appear simple at large scales, they can be locally heterogeneous, laterally discontinuous, and challenging to understand. Heterogeneity defines the vertical and lateral regional variability in material properties, resulting from changes in material texture and composition. The depositional lithofacies can change as a function of post-depositional processes of diagenesis, interaction with living organisms, thermally-activated geochemistry, and movement of mineral-laden fluids. The changes may be subtle, but sufficient for some of these lithofacies to develop considerably better reservoir potential than others. In unconventional oil and gas producing shales, building block lithofacies are primarily variations of argillaceous, siliceous, calcareous, and transitional mixtures of these end-member matrix compositions. In addition, these facies vary in depositional texture, organic content, and clay and kerogen maturation.

The economic viability of unconventional reservoirs is affected by three elements: heterogeneity; Reservoir Quality (RQ); and Completion Quality (CQ). Reservoir Quality is defined by the combination of properties leading to hydrocarbon storage (including interstitial and adsorbed components) and producibility, including hydrocarbon-filled porosity, pore-fluid saturations, effective permeability, organic content, and pore pressure. Completion Quality is defined by the combination of properties leading to surface area contacting the reservoir during production, including fracture containment, fracture complexity, retention of fracture area, and retention of fracture conductivity. Conditions affecting the loss of fracture area and fracture conductivity relevant to completion quality include rock-fluid sensitivity, proppant transport, proppant embedment or crushing, loss of fracture face permeability by imbibition, water retention, and solids production.

Oil and gas companies often act based on determinations made regarding RQ and CQ. The usual workflow to determine RQ and CQ involves analyses of logs for basic inferences on RQ and CQ. More detailed data becomes available later after testing core samples in the laboratory, and this testing provides a more accurate picture of RQ and CQ.

Computed Tomography (CT) of rock core samples from test wells has been used to determine properties of the rock core samples as well as characteristics of the well. CT scanning provides a digital record of the core sample before any de-tubing, slabbing, or invasive testing is done on the core sample. These measurements can be used for a variety of purposes, including facilitating sample selection (see, for example, U.S. Pat. No. 8,571,799, incorporated herein by reference) and improving geologic depositional models. Such CT scanning may employ single energy or dual energy techniques. Dual energy CT scans can be used to obtain high resolution measurements of bulk density and effective atomic number. Both bulk density and effective atomic number are essentially compositional measurements, and they provide insight into RQ. However, as will be described in greater detail herein, CT data can also be used, along with other high resolution core scanning measurements, to provide insight into rock texture and CQ.

### SUMMARY

A workflow is provided that characterizes the hydraulic fracturability of a core sample of reservoir rock based on CT data derived from CT scanning of the core sample as well as non-CT data derived from other tests and measurements performed on the core sample. The CT data and the non-CT data can be derived as a function of axial position in the core sample. The workflow employs correlation of the CT data and the non-CT data to generate a Heterogeneous Rock Analysis (HRA) model of the core sample. The HRA model identifies one or more rock units within the core sample. The workflow further derives hydraulic fracturability index values for the rock unit(s) of the core sample. The hydraulic fracturability index value for a given rock unit provides an indication of the fracturability of the given rock unit by hydraulic fracturing methods. In one embodiment, the hydraulic fracturability index value is a real value between 0 (which represents poor fracturability of the given rock unit by hydraulic fracturing methods) and 1 (which presents good fracturability of the given rock unit by hydraulic fracturing methods).

Both CQ and RQ factors can be used to infer the hydraulic fracturability index. CQ and RQ are two factors that can be used to determine the economic viability of an unconventional reservoir. CQ is a predictive attribute that can help predict successful reservoir stimulation through hydraulic fracturing. The assessment of CQ typically addresses the contact of surface area of the reservoir, including fracture containment and complexity, and the preservation of surface area and fracture conductivity during production. RQ is a predictive attribute that can help predict the ability of the reservoir to produce hydrocarbons economically after hydraulic fracture stimulation. The assessment of RQ typically addresses a number of properties of the reservoir rock, including hydrocarbon filled porosity, water saturation, permeability, mineral content and maturation, organic content and maturation, and pore pressure. The hydraulic fracturability index value(s) for the rock unit(s) of a core sample can be evaluated to select one or more rock units within a core sample for additional testing and analysis to derive CQ and/or RQ of the selected rock units of the core sample.

The hydraulic fracturability index value(s) for the rock unit(s) of a core sample can be propagated using the HRA method to other locations in the same well from which the core sample was obtained as well as locations in other surrounding wells, where such locations have rock properties that are statistically similar to the rock properties for the rock unit(s) of the core sample.

### BRIEF DESCRIPTION OF THE DRAWINGS

FIG. 1 is a schematic illustration of a workflow for generating an HRA model of a core sample that identifies one or more rock units within the core sample and deriving hydraulic fracturability index values for each rock unit of the core sample in accordance with the present disclosure.

FIG. 2 is an illustration of a window of a graphical user interface that presents CT and other data of a core sample that has been subject to CT scanning.

FIG. 3 is an illustration of resistivity data of a core sample, showing a fracture and parameters of the fracture that can be used to determine dip angle of the fracture.

FIG. 4 is an illustration of an embodiment of a core holder.

### DETAILED DESCRIPTION

As used herein, the term “core sample” means a rock sample obtained downhole from a reservoir, which is intended to be representative of the rock formation at the downhole location where the rock sample was obtained. The core sample can be cylindrical in shape extending along an axial direction, and obtained during or after drilling a well through the reservoir. Cores can be full-diameter cores (that is, they are nearly as large in diameter as the drill bit) taken at the time of drilling, or sidewall cores (generally 0.9 inch (23 mm) or 1.5 inches (38 mm) in diameter) taken after the borehole has been drilled.

As used herein, the term “rock unit” refers to a contiguous region of a core sample that has statistically uniform or similar properties relative to other regions of the core sample. The properties of interest that differentiate between rock units in a core sample can pertain to fluid movement and fluid storage capacity, for example, or many other factors. Also, there is statistical variation within each individual rock unit.

As used herein, the term “heterogeneity” refers to the vertical and lateral variability in properties of reservoir rock, which can result from changes in material texture and composition over the reservoir rock, for example.

As used herein, the term “interface” refers to a distinct change in the rock character spatially. Interfaces can be weaker or stronger zones of limited thickness. As used herein, the term “fracture” refers to a surface of breakage (or potential breakage) within the rock. Fractures may be natural or induced by coring or core handling processes. Natural fractures may be filled (healed), unfilled, or partially filled.

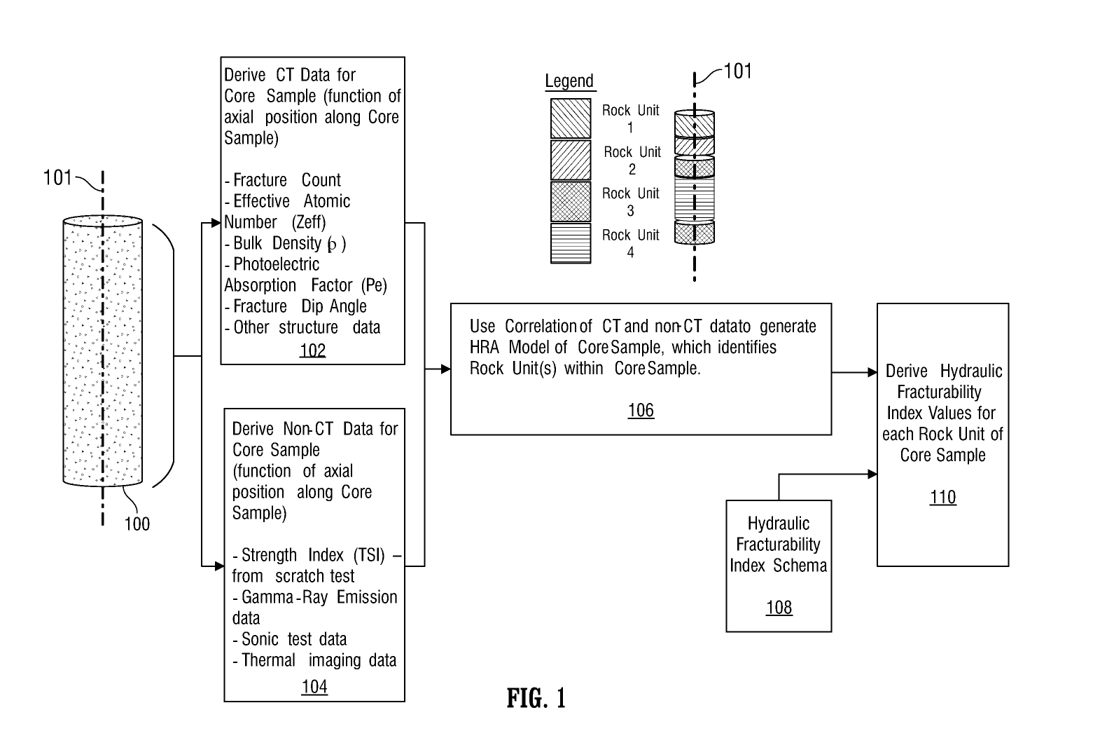

As will be described in greater detail below, and shown schematically in FIG. 1, properties of a core sample 100 measured by various methods are used to generate hydraulic fracturability index values for one or more rock units of the core sample 100. More specifically, in block 102, CT data is derived from CT scanning of a core sample 100. The CT data can be derived as a function of axial position in the core sample 100. In one embodiment, the CT data can include a fracture count, effective atomic number (Z_{eff}), bulk density (ρ), photoelectric absorption factor (Pe), fracture dip angle, and possibly other structure data for different axial positions in the core sample 100.

In block 104, non-CT data is derived from other tests and measurements performed on the core sample 100. The non-CT data can be derived as a function of axial position in the core sample 100. In one embodiment, the non-CT data can include a strength index (TSI) from a scratch test, gamma-ray emission data, sonic test data, or thermal imaging test data for different axial positions in the core sample 100. Non-CT data may also be derived from high resolution downhole logs such as resistivity logs produced by an FMI™ tool available from Schlumberger Technology Corporation of Sugar Land, Tex., USA.

In block 106, the CT data of 102 and the non-CT data of 104 are correlated to generate an HRA model of the core sample 100. The HRA model identifies one or more rock units within the core sample 100. A schematic representation of the HRA model of the core sample 100 is shown in FIG. 1 with five rock units disposed over the axis 101 of the core sample 100 according to the legend shown.

In block 110, hydraulic fracturability index (HFI) values are derived for the rock unit(s) of the core sample 100 as represented by the HRA model of the core sample 100 generated in block 106. The derivation of the hydraulic fracturability index values is based on a predefined hydraulic fracturing index schema 108 as shown. The hydraulic fracturability index value for a given rock unit provides an indication of the fracturability of the given rock unit by hydraulic fracturing methods. In one embodiment, the hydraulic fracturability index value is a real value between 0 (which represents poor fracturability of the given rock unit by hydraulic fracturing methods) and 1 (which presents good fracturability of the given rock unit by hydraulic fracturing methods). It should be noted that the HFI value can have some statistical variation within a particular rock unit.

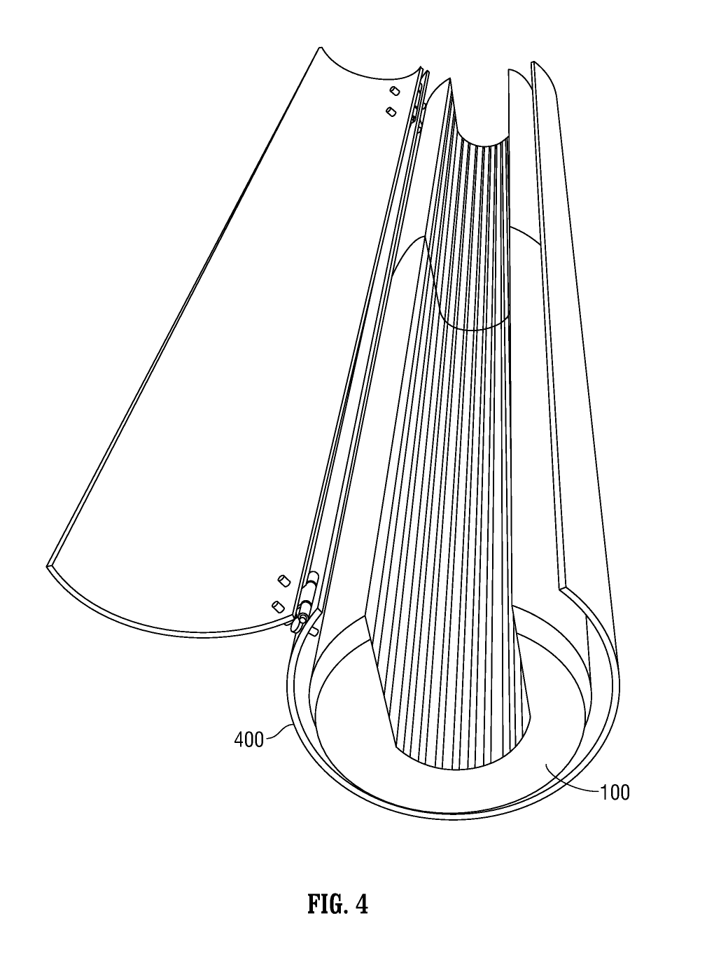

The CT data of block 102 can be derived from tomographic images of the core sample acquired using a commercially available CT scanner (such as a multi-slice, helical CT scanner that is configured to carry out single energy or dual energy imaging methods). The tomographic images can be many cross-sectional two-dimensional slices through the core sample 100, as described in “Whole Core CT Scanning from Core Flow Services”, Schlumberger, 2013, available from http://www.slb.com/˜/media/Files/core_pvt_lab/product sheets/coreflow_wholecore_ps. aspx, the entire contents of which are incorporated herein. Slices may be spaced as closely together as half a millimeter and may extend the length of the whole core sample. A three-dimensional volume of the core sample 100 can be viewed using a software application that animates the result, sequentially showing each axial slice. The axial slices may also be reconstructed to show views within the core sample along the longitudinal coronal plane and a sagittal plane that is perpendicular to the longitudinal plane. Additionally, the axial slices can be used to construct a two-dimensional image of the near-outer surface of the core sample. Such an image is termed a cylindrical unwrap image, because it is a two-dimensional flat image of the unwrapped near-outer surface of the cylindrical core. The tomographic images can be analyzed in an automated manner to provide quantitative data on rock texture, e.g., natural fractures, drilling induced fractures, interfaces, and rock stratifications. When a core sample is scanned during a CT scan, it may be held in a core holder to orient the core sample with respect to a CT scanner. FIG. 4 shows one possible embodiment of a core holder 400 made from aluminum and is shown without a core sample 100 inside an axial cavity formed in a foam liner of the core holder 400. In use, the core sample 100 is placed inside a respective core holder 400 forming an assembly and the assembly is placed in a computed tomography (CT) scanner for CT scanning of the core sample. A core sample may also be subject to CT scanning without being placed in a core holder.

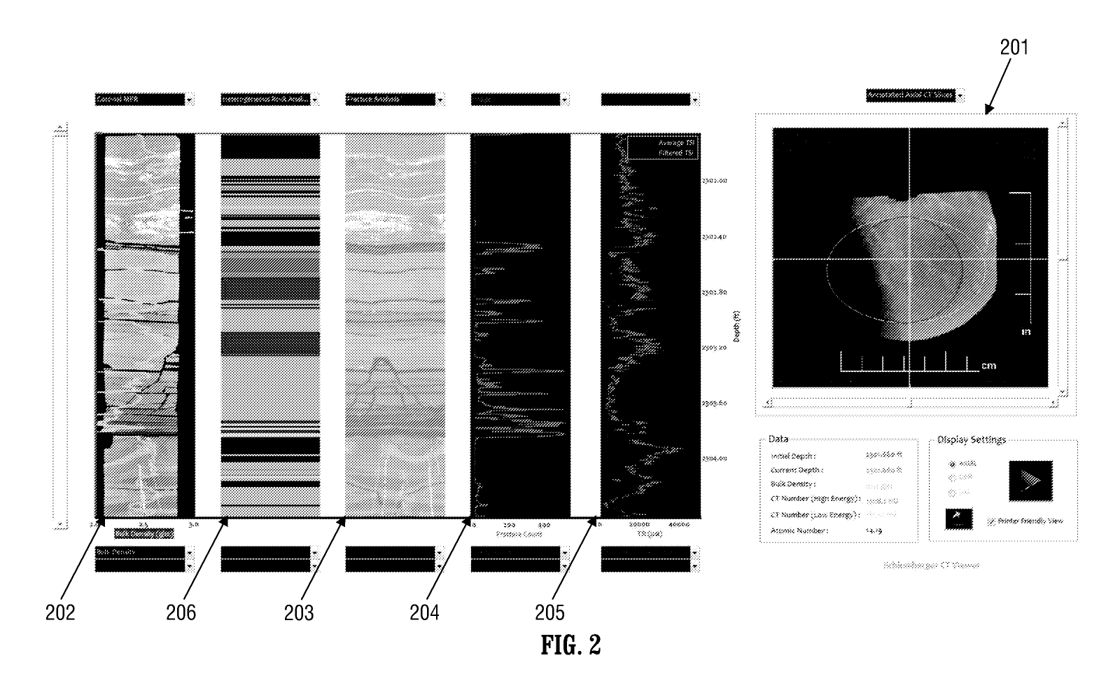

As noted above, the three-dimensional image of the core sample can be viewed using a graphical user interface (software application) that animates the result, sequentially showing each slice. An example illustration of such a graphical user interface is shown in FIG. 2. More specifically, region 201 of the graphical user interface shows a two-dimensional tomographic image of the core sample for a slice transverse to the axis of the core sample. Region 202 of the graphical user interface shows a two-dimensional tomographic image of the core sample for a longitudinal slice parallel to the axis of the core sample along with measurements of bulk density in blue as a function of axial position (as measured in block 102). Region 203 of the graphical user interface shows a two-dimensional image produced by unwrapping a cylindrical outer view of the core sample along with fractures highlighted in red. Region 204 of the graphical user interface shows a log (histogram) of fracture count in the image of region 203 as a function of axial position of the core sample (as measured in block 102). Region 205 of the graphical user interface shows a log of the strength index TSI as a function of axial position of the core sample (measured in block 104). Region 206 of the graphical user interfaces shows the rock units of the core sample as represented by the HRA model of the core sample 100 (as generated in block 106).

As noted above, the CT data of the core sample derived in block 102 can include a fracture count that is measured as a function of axial position of the core sample. The fracture counts quantify the amount and spacing of interfaces and/or fractures in the core sample. In one embodiment, the fracture count over different axial positions of the core sample can be derived by constructing a two-dimensional image (referred to a “cylindrical unwrapped image”) by unwrapping a cylindrical outer view of the core sample (e.g., region 203 of FIG. 2) and subjecting the cylindrical unwrapped image to image processing techniques that discriminate between portions of the image that represent an interface or fracture in the core sample. The image processing techniques applied to the cylindrical unwrapped image may be done at a pixel level and a thresholding algorithm may be used to discriminate whether or not a scanned pixel is or is not accounted for as part of an interface or fracture. If the pixel is determined to be part of a fracture and/or interface, it is “counted” as part of the fracture count at the corresponding axial position of the core sample. The end result of such image processing produces a log (histogram) of fracture count as a function of axial position of the core sample, which is depicted in region 204 of the graphical user interface of FIG. 2.

In order to discriminate whether or not a scanned pixel is or is not accounted for as part of a feature (an interface or fracture), the image processing may employ a grayscale thresholding algorithm that determines whether the grayscale level of the scanned pixel (or a group of pixels) satisfies a predefined threshold that is representative of the feature in the core sample. The scanning may be done, for example, by scanning transverse slices defined in the cylindrical unwrapped image along the length of the cylindrical unwrapped image. The slices may be closely spaced together (at pixel resolution) to improve the resolution of the scanning. The number of features found in each slice represents the fracture count at a particular axial position. More specifically, the pixels identified as representative of part of a feature are included as making up the fracture counts and the amount of fracture counts per unit depth along the core axis can be termed the “fracture intensity” associated with the axial position of the core sample. Other imaged data, such as FMI™ resistivity log data, may be substituted for CT data to carry out such an analysis.

Note that localized areas of the core sample with higher fracture intensity correspond to axial locations in the core (and therefore the well from which the core was obtained) where fracture complexity is expected or where fracture height containment during hydraulic fracturing is expected. As will be described in greater detail below, the fracture counts can be partly used to derive the hydraulic fracturability index values for the rock units of the core sample.

As noted above, the CT data of the core sample derived in block 102 can also include other rock properties (such as bulk density ρ, effective atomic number Z_{eff}, photoelectric absorption factor Pe, or fracture dip angle) that are measured as a function of axial position in the core sample.

With respect to measuring bulk density and the effective atomic number Z_{eff}, the CT scanner may operate in a dual-energy mode at two X-ray energies. During the CT process, the attenuation of an x-ray beam is measured as it passes through a sample material. The attenuation coefficient μ is defined as the fractional decrease in x-ray intensity per unit length of the material, and is a function of atomic number and bulk density of the material and the x-ray energy. Generally, the linear attenuation coefficient is normalized to that of a standard material (e.g., water), and is defined as the CT number of the material

$$CT = μ material - μ standard μ CT = \frac{{μ}_{material} - {μ}_{standard}}{{μ}_{standard}} \cdot K \qquad ( 1 )$$

where K is a scaling factor. The x-ray attenuation coefficient μ may be represented by Eq. 2 below:

μ=α+bρ+cU. (2)

In Eq. 2, ρ is the average bulk (or electron) density, and U is a function of average bulk density, ρ, and average atomic number, Z, as represented by Eq. 3 below:

U=ρPe (3)

In Eq. 3,

$$Pe = {( \frac{z}{10} )}^{m} = photoelectric absorption factor ,$$

and m is a constant ranging in value from 3 to 4. If a mixture of atomic species is present (as is the case when scanning rock material), then Pe is proportional to the effective atomic number, Z_{eff}, rather than Z.

For two energy levels (E1 and E2) and three known standards (e.g., quartz denoted with subscript A, teflon with subscript B, and aluminum with subscript C), by combining Eqs. 1 and 2, one can write a new system of equations (4) as shown below to solve for respective values of bulk density, ρ, and U (to determine atomic number Z) for each standard:

CT_{Ai}=aᵢ+bᵢρ_{A}+cᵢU_{A}

CT_{Bi}=aᵢ+bᵢp_{B}+cᵢU_{B}

CT_{Ci}=aᵢ+bᵢρ_{c}+cᵢU_{C} (4)

Then, for the core sample, the bulk density ρ and U (to determine effective atomic number Z_{eff}) may be obtained by solving the system of equations (5) below when the core sample is scanned with a CT scanner at the same energy levels (E1 and E2), as the standards:

CT₁=a₁+b₁ρ+c₁U

CT₂=a₂+b₂ρ+C₂U (5)

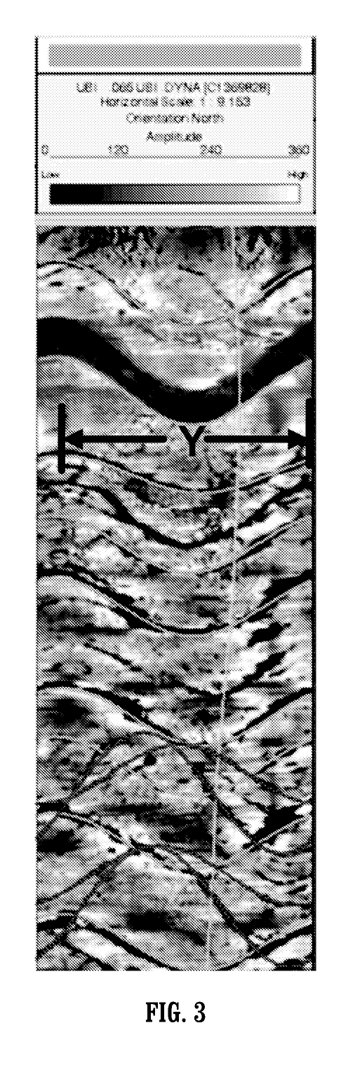

With respect to fracture dip angle, the cylindrical unwrapped image of the core sample can be subject to image processing techniques that identify fractures or other features by connecting approximately linear features in the core to form a sinusoid on the cylindrically unwrapped resistivity image as shown in FIG. 3. Such an image may also be comprised of CT or other suitable data. The sinusoid can be analyzed to find the fracture dip angle according to Eq. 6, for example, as follows:

Fracture Dip Angle=arctan(Y/D), (6)

In this case, the parameter Y is the peak to peak distance of the sinusoid (in millimeters), and the parameter D is the diameter of the core sample (in millimeters). Also, fracture orientation may be determined by the azimuth of the sinusoid troughs, read from the direction scale at the top of the image. The description of natural and induced fractures in the rock formation from which the core was obtained can be used as inputs to, for example, the Mangrove application for hydraulic fracturing simulation design/evaluation (see U.S. Pat. Nos. 8,412,500 and 8,571,843 and U.S. Patent Application Publication 2013/0319657, all incorporated herein by reference) in the PETREL™ shared earth model software available from Schlumberger Technology Corporation of Sugar Land, Tex., USA.

As noted above, the non-CT data of the core sample derived in block 104 can include a strength index, TSI, which can be determined from high resolution scratch (compositional) measurements of the core sample at multiple axial positions of the core sample. The scratch measurements can be carried out using scratch test equipment available from TerraTek, Inc. of Salt Lake City, Utah, USA, a subsidiary of Schlumberger Technology Corporation. Such measurements are described in U.S. Pat. No. 8,234,912, incorporated herein by reference. The scratch measurements and resulting non-CT data can include other measures of rock composition or texture.

The non-CT data of the core sample derived in block 104 can also include data measured by gamma ray emission testing, sonic testing, and/or thermal imaging of the core sample, for example. With respect to the gamma ray emission testing, all rocks contain natural radioactive material, but shales have much higher gamma emissions than others such as sandstone or limestone. Gamma ray emission testing measures the gamma rays emitted by the natural radioactive material of the core sample.

As noted above for block 106, the CT data (e.g., bulk density, effective atomic number, fracture count, photoelectric absorption factor Pe, fracture dip angle) for the different axial positions of the core sample can be correlated to the non-CT data (e.g., strength index TSI, gamma ray emission data, sonic test data, thermal imaging data) for the corresponding axial positions of the core sample to derive an HRA model of the core sample. The HRA model of the core sample defines the non-redundant rock units within the core sample, each with a statistically distinct combination of material properties. Details of exemplary HRA models are described in U.S. Pat. Nos. 7,983,885 and 8,200,465, which are incorporated herein by reference in their entireties.

The HRA model of the core sample provides a mathematically precise, objective, and robust methodology for rock classification based on rock behavior and material properties. The HRA model of the core sample accounts for thickness, vertical stacking patterns and spatial distribution of rock classes with similar behavior and similar material properties.

The HRA model of the core sample can also provide a quantitative measure of the similarity (or compliance) between the rock units in the core sample and those identified in other core samples in the same well and other wells. The analysis of similarity provides a regional measure of the confidence in the model and a reference for evaluating cost/benefit conditions for improving the reference model with additional measurements (core, logs, and seismic) to reduce the uncertainty. The HRA model of the core sample can also provide high resolution mapping of the cyclic depositional units and their similarities and dissimilarities based on material properties. This information is fundamental for core-based geology and sedimentology studies of the well.

As noted above for block 110, hydraulic fracturability index values are derived for the rock unit(s) of the core sample 100 as represented by the HRA model of the core sample. The hydraulic fracturability index values can be used to distinguish good from poor hydraulic fracturability zones along the axial length of the core sample.

The derivation of the hydraulic fracturability index value for a given rock unit is based upon a predefined hydraulic fracturability index schema (“HFI Schema”) 108. Table 1 below illustrates an embodiment of an HFI Schema for the determination of the hydraulic fracturability index value in a hydrocarbon-containing pay zone according to the present disclosure. A pay zone is a zone in a rock formation that contains hydrocarbon-bearing fluids. A pay zone is distinguished from a boundary zone, which is a zone in a rock formation that contains little or no hydrocarbon content. Note that in a hydrocarbon-containing pay zone, rocks with greater hydraulic fracturability are assigned higher HFI values (in the scale from 0 to 1) as compared to rocks with less hydraulic fracturability.

TABLE 1

| | | | | Parameter | |
|---|---|---|---|---|---|
| | Parameter | Parameter | Parameter | Constraints | Parameter |
| | Constraints | Constraints for | Constraints for | for | Constraints for |
| Measured | for | HFI value = | HFI value = | HFI value = | HFI value = |
| Parameter for | HFI value = 0 | 0-0.25 | 0.25-0.50 | 0.75-1.0 | 1.0 |
| Rock Unit | (Poor-Red) | (Orange) | (Yellow) | (Green) | (Good-Blue) |
| TSI (psi) | <1000 | 1000-1500 | 1500-2000 | 2000-2500 | 2500-3000 |
| TSI (psi) | >10,000 | 8250-10,000 | 6500-8250 | 4750-6500 | 3000-4750 |
| CT Z_{eff} | 15.0-15.2 | 14-15 | 13-14 | 12-13 | <12 |
| CT Z_{eff} | 15.2-16.0 | 16-17 | 17-18 | 18-19 | >19 |
| CT_{rhoB} (g/cc) | <2.2 | 2.2-2.3 | 2.3-2.4 | 2.4-2.5 | 2.5-2.6 |
| CT_{rhoB} (g/cc) | >3.0 | 2.9-3.0 | 2.8-2.9 | 2.7-2.8 | 2.6-2.7 |
| CT dipping | <50 | 50-100 | 100-150 | 150-200 | >200 |
| fracture count | | | | | |

CT data is averaged on each axial slice in a circular or ellipsoid region of that slice. TSI data is averaged over width and depth of a groove cut into the (slabbed or outer) surface of the core. The “dipping” fracture count is a count of interfaces/features/fractures, running along the core axis, that are not orthogonal to the core axis. It will be appreciated that most core samples are cut approximately vertically through a stack of sedimentary layers, with horizontally oriented bedding, and this assumption is carried through in this description for simplicity, unless mentioned otherwise. In that regard, if the core is dipping, i.e., cored such that the bedding layers are inclined at an angle with respect to the core axis, a dipping fracture count would be defined as being non-parallel to the dominant bedding orientation.

Note that the exemplary HFI Schema of Table 1 maps certain ranges (constraints) of the CT-based and non-CT based parameters (strength index TSI, effective atomic number, bulk density, and dipping fracture count) to five color-coded different hydraulic fracturability index values (from 0 to 1). For purposes of the example shown in Table 1, the range of possible hydraulic fracturability index values has been divided into five color-coded levels, with each level including ranges of parameter values associated with the respective level. The leftmost (red) level includes parameter values individually indicative of poor hydraulic fracturability in the pay zone, which is undesired in the pay zone. The rightmost (blue) level includes parameter values individually indicative of good hydraulic fracturability in the pay zone, which is desired in the pay zone. While five levels are shown in the example in Table 1, it will be appreciated that for other embodiments, the number of levels may be more or less than five. It will also be appreciated from the example in Table 1, that at each level, the particular parameter value may be defined either by a single data range (high vs. low values) or by two data ranges to account for a case where the value has an optimum. For example, values of TSI shown in the red level are less than 1,000 psi and greater than 10,000 psi. However, values of TSI in the blue level are in a range between 2500 and 4750 psi. In addition, the numerical values defining the limits of each colored level may be adjusted. Thus, Table 1 is merely illustrative.

Also note that the exemplary HFI Schema of Table 1 employs hydraulic fracturability index values in the range from 0 to 1, with a hydraulic fracturability index value of 0 indicating poor hydraulic fracturability and a hydraulic fracturability index value of 1 indicating good hydraulic fracturability. The intermediate hydraulic fracturability index values between 0 and 1 represent increasingly better hydraulic fracturability.

The measurements of the strength index TSI, effective atomic number (CT Z_{eff}), bulk density (CT_{rhoB}), and dipping fracture count for each rock unit of the HRA model of block 106 can be compared with parameter constraints of the HFI Schema of Table 1 to assign a corresponding hydraulic fracturability index value. For example, a pay zone rock unit with a strength index TSI of 1200, an effective atomic number (CT Z_{eff}) of 14, a bulk density (CT_{rhoB}) of 2.2 and a dipping fracture count of 60 would be assigned a hydraulic fracturability index value of (0-0.25) or Orange according to the HFI Schema of Table 1. The HFI value may be displayed stepwise, or may be interpolated for a smoothly varying index as a function of supplied measurements along the core.

It is also contemplated that measurements of the strength index TSI, effective atomic number (CT Z_{eff}), bulk density (CT_{rhoB}), and dipping fracture count for a pay zone rock unit of the HRA model may not correspond to the parameter constraints defined for a single level (i.e., a single column) of the HFI Schema of Table 1. For example, a pay zone rock unit of the HRA model may have a TSI value of 500 psi (satisfied by the TSI constraints for the column corresponding to the HFI value of 0 or Poor-Red) and a dipping fracture count of 125 (satisfied by the fracture constraints for the column corresponding to the HFI value of 0.25-0.50 or Yellow. To take this variability into account, the pay zone HFI schema can be adapted by assigning weights to respective ranges for each one of the four parameters (TSI, CT Z_{eff}, CT_{rhoB}, and dipping fracture count), averaging the weights for the matching parameter range for the four parameters (TSI, CT Z_{eff}, CT_{rhoB}, and dipping fracture count) to give a weighted average, and assigning HFI values to the possible ranges of the weighted averages.

For example, a limited set of possible level combinations and their resulting HFI values are identified in Table 2 below for an exemplary pay zone HFI schema.

TABLE 2

| | | | | | | | | | | | | | |
|---|---|---|---|---|---|---|---|---|---|---|---|---|---|
| TSI (psi) | <1000 | 1000-1500 | 1500-2000 | 2000-2500 | 2500-3000 | R_{TSI} | R_{TSI} | R_{TSI} | B_{TSI} | O_{TSI} | G_{TSI} | R_{TSI} | R_{TSI} |
| | >10,000 | 8250-10,000 | 6500-8250 | 4750-6500 | 3000-4750 | | | | | | | | |
| | (R_{TSI}- | (O_{TSI}- | (Y_{TSI}-- | (G_{TSI}- | (B_{TSI}- | | | | | | | | |
| | weight | weight | weight | weight | weight | | | | | | | | |
| | of 1) | of 2) | of 3) | of 4) | of 5) | | | | | | | | |
| CT Z_{eff} | 15-16 | 14-15 | 13-14 | 12-13 | <12 | R_{Zeff} | R_{Zeff} | R_{Zeff} | B_{Zeff} | O_{Zeff} | G_{Zeff} | O_{Zeff} | Y_{Zeff} |
| | (R_{Zeff}- | 16-17 | 17-18 | 18-19 | >19 | | | | | | | | |
| | weight | (O_{Zeff}- | (Y_{Zeff}- | (G_{Zeff}- | (B_{Zeff}- | | | | | | | | |
| | of 1) | weight | weight | weight | weight | | | | | | | | |
| | | of 2) | of 3) | of 4) | of 5) | | | | | | | | |
| CT_{rhoB} | <2.2 | 2.2-2.3 | 2.3-2.4 | 2.4-2.5 | 2.5-2.6 | R_{BD} | Y_{BD} | B_{BD} | R_{BD} | G_{BD} | O_{BD} | G_{BD} | G_{BD} |
| (g/cc) | >3.0 | 2.9-3.0 | 2.8-2.9 | 2.7-2.8 | 2.6-2.7 | | | | | | | | |
| | (R_{BD}- | (O_{BD}- | (Y_{BD}- | (G_{BD}- | (B_{BD}- | | | | | | | | |
| | weight | weight | weight | weight | weight | | | | | | | | |
| | of 1) | of 2) | of 3) | of 4) | of 5) | | | | | | | | |
| CT | <50 | 50-100 | 100-150 | 150-200 | >200 | B_{FC} | Y_{FC} | B_{FC} | R_{FC} | G_{FC} | O_{FC} | B_{FC} | B_{FC} |
| dipping | (R_{FC}- | (O_{FC}- | (Y_{FC}- | (G_{FC}- | (B_{FC}- | | | | | | | | |
| fracture | weight | weight | weight | weight | weight | | | | | | | | |
| count | of 1) | of 2) | of 3) | of 4) | of 5) | | | | | | | | |
| Resulting | Weighted | | | | | | | | | | | | |
| HFI value | Average | | | | | | | | | | | | |
| for pay | WA of 1 | WA of 2 | WA of 3 | WA of 4 | WA of 5 | WA | WA | WA | WA | WA | WA | WA | WA |
| zone | | | | | | of 2 | of 2 | of 3 | of 3 | of 3 | of 3 | of 3 | of |
| | | | | | | | | | | | | | 3.25 |
| | Poor-R | O | Y | G | Good-B | O | O | Y | Y | Y | Y | Y | Y |
| | WA in | WA in | WA in | WA in | WA in | | | | | | | | |
| | range of | range of | range | range | range | | | | | | | | |
| | 1-1.5 | 1.5-2.5 | of 2.5-3.5 | of 3.5-4.5 | of 4.5-5 | | | | | | | | |
| | HFI | HFI | HFI | HFI | HFI | | | | | | | | |
| | value = 0 | value = | value = | value = | value = 1 | | | | | | | | |
| | | 0-.25 | .25-.50 | .75-1 | | | | | | | | | |

For example, for one combination in Table 2, in the case that the measured TSI value falls in the range of <1000 psi or >10,000 psi (R_{TSI}—weight of 1), the measured CT Z_{eff} falls in the range of 15-16 (R_{Zeff}—weight of 1), the measured CT_{rhoB} falls in the range of 2.3-2.4 g/cc or 2.8-2.9 g/cc (Y_{BD}—weight of 3), and the measured dipping fracture count falls in the range of 100-150 (Y_{FC}—weight of 3), the weighted average is derived (1+1+3+3)/4=8/4=2, which corresponds to the Orange level “O” whose weighted average is in range of 1.5-2.5 and assigned an HFI value=0-0.25.

Table 3 below illustrates an embodiment of an HFI schema for the determination of the hydraulic fracturability index value of a rock unit that is present in a boundary zone according to the present disclosure. Note that in a boundary zone, rock with greater hydraulic fracturability is assigned a higher HFI value (in the scale from 0 to 1) as compared to rock with less hydraulic fracturability.

TABLE 3

| | | Parameter | Parameter | Parameter | Parameter |
|---|---|---|---|---|---|
| | Parameter | Constraints | Constraints | Constraints | Constraints |
| | Constraints | for | for | For | for |
| Measured | for | HFI value = | HFI value = | HFI value = | HFI value = |
| Parameter for | HFI value = 0 | 0-0.25 | 0.25-0.50 | 0.75-1.0 | 1.0 |
| Rock Unit | (Poor-Red) | (Orange) | (Yellow) | (Green) | (Good-Blue) |
| TSI (psi) | 3000-4750 | 4750-6500 | 6500-8250 | 8250-10,000 | >10,000 |
| CT Z_{eff} | <12 | 12-13 | 13-14 | 14-15 | 15-15.2 |
| CT Z_{eff} | >19 | 18-19 | 17-18 | 16-17 | 15.2-16 |
| CT_{rhoB} (g/cc) | <2.7 | 2.7-2.8 | 2.8-2.9 | 2.9-3 | >3.0 |
| CT | <50 | 50-100 | 100-150 | 150-200 | >200 |
| horizontal | | | | | |
| fracture count | | | | | |

Note that exemplary HFI schema of Table 3 maps certain ranges (constraints) of the CT-based and non-CT-based parameters (strength index TSI, effective atomic number, bulk density, and horizontal fracture count) to five color-coded different hydraulic fracturability index values (from 0 to 1). For purposes of the example shown in Table 3, the range of possible hydraulic fracturability index values has been divided into five color-coded levels, with each level including ranges of parameter values associated with the respective level. The leftmost level, red level, includes parameter values individually indicative of poor hydraulic fracturability which is undesired in the boundary zone. The rightmost level, blue level, includes parameter values individually indicative of good hydraulic fracturability, which is desired in the boundary zone. While five levels are shown in the example in Table 3, it will be appreciated that for other embodiments, the number of levels may be more or less than five. It will also be appreciated from the example in Table 3, that at each level, the particular parameter value may be defined either by a single data range (high vs. low values) or by two data ranges to account for a case where the value has an optimum.

Also note that the exemplary HFI schema of Table 3 employs hydraulic fracturability index values in the range from 0 to 1, with a hydraulic fracturability index value of 0 indicating poor hydraulic fracturability and a hydraulic fracturability index value of 1 indicating good hydraulic fracturability. The intermediate hydraulic fracturability index values between 0 and 1 represent increasingly better hydraulic fracturability.

The measurements of the strength index TSI, effective atomic number (CT Zen), bulk density (CT_{rhoB}), and horizontal fracture count for each rock unit of the HRA model of block 106 can be compared with parameter constraints of the HFI schema of Table 3 to assign a corresponding hydraulic fracturability index value. For example, a boundary zone rock unit with a strength index TSI of 8000, an effective atomic number (CT Z_{eff}) of 14, a bulk density (CT_{rhoB}) of 2.8 and a horizontal fracture count of 100 would be assigned a hydraulic fracturability index value of (0.25-0.50) or Yellow according to the HFI schema of Table 3.

It is also contemplated that measurements of the strength index TSI, effective atomic number (CT Z_{eff}), bulk density (CT_{rhoB}), and horizontal fracture count for a boundary zone rock unit of the HRA model may not correspond to the parameter constraints defined for a single level (i.e., a single column) of the HFI schema of Table 3. To take this variability into account, the boundary zone HFI schema can be adapted by assigning weights to respective ranges for each one of the four parameters (TSI, CT Z_{eff}, CT_{rhoB}, and horizontal fracture count), averaging the weights for the matching parameter range for the four parameters (TSI, CT Z_{eff}, CT_{rhoB}, and horizontal fracture count) to give a weighted average, and assigning HFI values to the possible ranges of the weighted averages.

For example, a limited set of possible level combinations and their resulting HFI values are identified in Table 4 below for an exemplary boundary zone HFI schema.

TABLE 4

| | | | | | | | | | | | | | |
|---|---|---|---|---|---|---|---|---|---|---|---|---|---|
| TSI (psi) | 3000-4750 | 4750-6500 | 6500-8250 | 8250-10,000 | >10,000 | R_{TSI} | R_{TSI} | R_{TSI} | B_{TSI} | O_{TSI} | G_{TSI} | R_{TSI} | R_{TSI} |
| | (R_{TSI}- | (O_{TSI}- | (Y_{TSI}-- | (G_{TSI}- | (B_{TSI}- | | | | | | | | |
| | weight of | weight | weight | weight | weight | | | | | | | | |
| | 1) | of 2) | of 3) | of 4) | of 5) | | | | | | | | |
| CT Z_{eff} | <12 | 12-13 | 13-14 | 14-15 | 15-16 | R_{Zeff} | R_{Zeff} | R_{Zeff} | B_{Zeff} | O_{Zeff} | G_{Zeff} | O_{Zeff} | Y_{Zeff} |
| | >19 | 18-19 | 17-18 | 16-17 | (B_{Zeff}- | | | | | | | | |
| | (R_{Zeff}- | (O_{Zeff}- | (Y_{Zeff}- | (G_{Zeff}- | weight | | | | | | | | |
| | weight of | weight | weight | weight | of 5) | | | | | | | | |
| | 1) | of 2) | of 3) | of 4) | | | | | | | | | |
| CT_{rhoB} | <2.7 | 2.7-2.8 | 2.8-2.9 | 2.9-3.0 | >3.0 | R_{BD} | Y_{BD} | B_{BD} | R_{BD} | G_{BD} | O_{BD} | G_{BD} | G_{BD} |
| (g/cc) | (R_{BD}- | (O_{BD}- | (Y_{BD}- | (G_{BD}- | (B_{BD}- | | | | | | | | |
| | weight of | weight | weight | weight | weight | | | | | | | | |
| | 1) | of 2) | of 3) | of 4) | of 5) | | | | | | | | |
| Horizontal | <50 | 50-100 | 100-150 | 150-200 | >200 | B_{FC} | Y_{FC} | B_{FC} | R_{FC} | G_{FC} | O_{FC} | B_{FC} | B_{FC} |
| fracture | (R_{FC}- | (O_{FC}- | (Y_{FC}- | (G_{FC}- | (B_{FC}- | | | | | | | | |
| count | weight of | weight | weight | weight | weight | | | | | | | | |
| | 1) | of 2) | of 3) | of 4) | of 5) | | | | | | | | |
| Resulting | Weighted | | | | | | | | | | | | |
| HFI value | Average | | | | | | | | | | | | |
| for pay | WA of 1 | WA of 2 | WA of 3 | WA of 4 | WA of 5 | WA | WA | WA | WA | WA | WA | WA | WA |
| zone | | | | | | of 2 | of 2 | of 3 | of 3 | of 3 | of 3 | of 3 | of |
| | | | | | | | | | | | | | 3.25 |
| | Poor-R | O- | Y- | G- | Good-B | O | O | Y | Y | Y | Y | Y | Y |
| | WA in | WA in | WA in | WA in | WA in | | | | | | | | |
| | range | range | range | range | range of | | | | | | | | |
| | of 1-1.5 | of 1.5-2.5 | of 2.5-3.5 | of 3.5-4.5 | 4.5-5.0 | | | | | | | | |
| | HFI | HFI | HFI | HFI | HFI | | | | | | | | |
| | value = 0 | value = | value = | value = | value = | | | | | | | | |
| | | 0-.25 | .25-.50 | .75-1.0 | 1.0 | | | | | | | | |

For example, for one combination in Table 4, in the case that the measured TSI value falls in the range of 3000-4750 psi (R_{TSI}—weight of 1), the measured CT Z_{eff} falls in the range of <12 or >19 (R_{Zeff}—weight of 1), the measured CT_{rhoB} falls in the range of 2.8-2.9 g/cc (Y_{BD}—weight of 3), and the measured horizontal CT fracture count falls in the range of 100-150 (Y_{FC}—weight of 3), the weighted average is derived (1+1+3+3)/4=8/4=2, which corresponds to the orange level 0 whose weighted average is in range of 1.5-2.5 and assigned an HFI value=0-0.25.

In general, a rock unit with high CQ will have larger fracture width, lower breakdown pressure, less solids production potential, good perforation tunnel stability, and good fracturability (higher fracture count) relative to a rock unit with low completion quality. Unless a detailed series of additional specific laboratory tests are performed on each distinct rock unit of a core sample, it is not possible to rigorously determine the completion quality based on fracture width, breakdown pressure, solids production potential, perforation tunnel stability, and fracturability. However, those properties may be inferred from some of the compositional and textural measurements discussed herein.

For example, high perforation tunnel stability in a rock unit can be inferred from a high TSI value. Also, good fracturability and increased fracture height containment potential may be inferred from rock units with high fracture counts. Lower (or optimal) breakdown pressures may be inferred from rock units with lower strength index TSI values. Also, larger fracture width in rock units may be inferred from lower strength index TSI values and lower bulk density (these often correlate with lower Young's modulus). Moreover, less potential for solids production may be inferred from rock units with higher strength index TSI values and lower fracture counts.

It is clear that some data trends (not included in Tables 1 or 3) may conflict with each other in terms of the inferences to be drawn therefrom. For example, with regard to solids production potential, it is possible to mitigate solids production by optimizing the pressure drawdown during production so that the measured parameter of solids production need not be included in the HFI schema in Table 1. Similarly, with regard to open perforations, premature perforation closure is usually not a significant issue when wells are completed relatively quickly after perforating, so a measurement of open perforations also need not be included in the HFI schema in Table 1.

Once all of the distinct rock units of a core sample have been assigned hydraulic fracturability index values, those values can be correlated with other well log data from the well of the core sample so that the properties for the core sample can be propagated in like manner to other portions of that well, as well as other nearby wells. In this way it is possible to generate additional information about pay zone locations and completion quality for an entire logging area of multiple wells. Because the HRA rock class discrimination is done quantitatively and is based on material behavior and material properties, the method avoids assuming that similar depositional environments are conducive to similar material properties. Given the intense post-depositional diagenetic processes in fine-grained, organic-rich mudstones, the similarity or dissimilarity of the “end-product-rock” material properties is defined more by the subtle post-depositional bio-geoscience exercise, and not, a priori, a depositional geology one.

After coring and laboratory testing is complete, the HRA rock classification may be used to facilitate the population and propagation of measured properties from the cored well to other wells in the region, and to facilitate the development of core-to-log and log-to-seismic property relationships. This is done on a rock class-by-rock class basis. The end result is a regional-scale earth model populated with measured properties required for robust numerical modeling.

It is therefore possible to identify and select certain core samples (and rock units within such core samples) for additional tests for determining RQ, CQ, and/or for determining good versus poor hydraulic fracturability as needed. This can avoid testing whole core samples and parts of core samples that are not relevant to the understanding of the pay and boundary zones of the reservoir. It is then possible to use log-based HRA to extrapolate the core-based properties across an entire logged well and to other similar wells (i.e., laterally located with respect to the logged well), allowing the operator to make early decisions on completion strategies.

There have been described and illustrated herein several embodiments of a method of determining a hydraulic fracturability index. While particular embodiments have been described, it is not intended that the invention be limited thereto, as it is intended that the invention be as broad in scope as the art will allow and that the specification be read likewise. Thus, while particular parameters affecting hydraulic fracturability have been disclosed, it will be appreciated that other factors and/or core profiling measurements may be considered as well. In addition, while particular types of core holders have been disclosed, it will be understood that other types of core holders may be used. Also, while dual energy CT scanning is used, it will be recognized that single energy CT scanning may also be used. It will therefore be appreciated by those skilled in the art that yet other modifications could be made to the provided invention without deviating from its scope as claimed.

What is claimed is:

1. A method of characterizing the hydraulic fracturability of a core sample of reservoir rock, the method comprising:
Obtaining computed tomography (CT) data derived from CT scanning of the core sample;
obtaining non-CT data derived from other tests and measurements performed on the core sample; and
correlating the CT data and the non-CT data to generate a model of the core sample, wherein the model of the core sample defines one or more rock units within the core sample.

2. The method according to claim 1, further comprising:
deriving a hydraulic fracturability index value for each rock unit of the core sample as defined by the model of the core sample, wherein the hydraulic fracturability index value for a given rock unit provides an indication of the fracturability of the given rock unit by hydraulic fracturing methods.

3. The method according to claim 1, wherein the CT data and the non-CT data are derived as a function of axial position in the core sample.

4. The method according to claim 1, wherein the model is a heterogeneous rock analysis (HRA) model.

5. The method according to claim 2, wherein the hydraulic fracturability index value for a given rock unit is a real value between 0 and 1.

6. The method according to claim 5, wherein the hydraulic fracturability index value for a given rock unit being 0 represents poor fracturability of the given rock unit by hydraulic fracturing methods and the hydraulic fracturability index value for a given rock unit being 1 represents good fracturability of the given rock unit by hydraulic fracturing methods.

7. The method according to claim 1, wherein the non-CT data includes properties related to rock strength and texture derived by scratch test measurements.

8. The method according to claim 1, wherein the CT data includes compositional and textural properties of the core sample, including at least one of measurements of bulk density, effective atomic number, fracture count, and fracture count intensity.

9. The method according to claim 1, further comprising:
obtaining a tomographic image of the core sample based on the CT data;
processing the tomographic image to determine areas of the image representative of natural fractures or interfaces in the core sample; and
determining a fracture or interface count by counting the fractures or interfaces as a function of position in the core sample.

10. The method according to claim 9, wherein the tomographic image is a cylindrical unwrap image of the core sample.

11. The method according to claim 9, wherein processing the tomographic image includes subjecting the image to grayscale thresholding to identify portions of the image indicative of fractures or interfaces in the core sample.

12. The method according to claim 1, wherein the model is based on analysis of a first property among the CT data and a second property among the non-CT data.

13. The method according to claim 12, wherein the first property among the CT data is CT measurements and the second property among the non-CT data is scratch test measurements.

14. The method according to claim 2, wherein the hydraulic fracturability index value for each rock unit of the core sample is based on a predefined hydraulic fracturability index schema.

15. The method according to claim 14, wherein the hydraulic fracturability index schema maps certain ranges of properties to different hydraulic fracturability index values.

16. The method according to claim 15, wherein the mapped properties include at least one of strength index, effective atomic number, bulk density, horizontal fracture count, and dipping fracture count.

17. The method according to claim 16, wherein weights are assigned to respective mapped properties and the hydraulic fracturability index value for each rock unit is a weighted average.

18. A method of characterizing the hydraulic fracturability of a core sample of reservoir rock, the method comprising:
Obtaining computed tomography (CT) data based on a CT scan of the core sample;
obtaining scratch test data for the core sample; and
generating a heterogeneous rock analysis model of the core sample based at least on the CT data and the scratch test data to identify statistically distinct rock units of the core sample.

19. The method according to claim 18, further comprising:
assigning a hydraulic fracturability index value to each identified rock unit based at least on the generated CT data and the obtained scratch test data.

20. The method according to claim 18, wherein the CT data includes fracture count intensity, bulk density, and effective atomic number.

21. The method according to claim 19, wherein the CT data includes a fracture count.

22. The method according to claim 19, wherein the CT data and the scratch test data are functions of axial position in the core sample.

23. The method according to claim 19, wherein the hydraulic fracturability index value for a given rock unit of the core sample provides an indication of the fracturability of the given rock unit by hydraulic fracturing methods.
